(19) United States
(12) Patent Application Publication
(10) Pub. No.: US 2010/0274109 A1
(43) Pub. Date: Oct. 28, 2010

(54) MEASUREMENT APPARATUS FOR HEART RATE VARIABILITY

(75) Inventors: WEI-CHIH HU, CHUNG LI (TW); CHAO-FENG CHANG, CHUNG LI (TW)

Correspondence Address: ROSENBERG, KLEIN & LEE, 3458 ELLICOTT CENTER DRIVE-SUITE 101, ELLICOTT CITY, MD, 21043, US

(73) Assignee: CHUNG YUAN CHRISTIAN UNIVERSITY, CHUNG LI (TW)

(21) Appl. No.: 12/726,820

(22) Filed: Mar. 18, 2010

(30) Foreign Application Priority Data
Apr. 28, 2009 (TW) 098114098

(51) Int. Cl. A61B 5/1455; A61B 5/0408

(52) U.S. Cl. 600/310; 600/515

(57) ABSTRACT

The present invention relates to a measurement apparatus for heart rate variability, which comprises an earpiece, a measurement module for photoplethysmographic (PPG) signal measuring an ear and producing a first physiological signal of a person under test, a measurement module for electrocardiographic (ECG) signal measuring a second physiological signal of the person under test, and a control and processing unit transmitting a sound signal to the earpiece. By playing the sound signal from the earpiece, the attention of the person under test can be detracted from the measurement module for PPG signal or the measurement module for ECG signal. Thereby, nervousness and impatience of the person under test can be eliminated, and hence the real heart rate variability of the person under test can be measured.

### FIELD OF THE INVENTION

The present invention relates to a measurement apparatus, and particularly to a measurement apparatus for heart rate variability.

### BACKGROUND OF THE INVENTION

Heart rate is the frequency at which the heart beats, and its unit is beats per minute (BPM). In 1981, Akselrod published a method for giving the characteristic power spectrum of heart rate variability (HRV) by fast Fourier transform, where the heart rate variability is the difference between each heartbeat interval, namely, the variations in heart rates or heartbeat intervals. The characteristic power spectrum of HRV corresponds to the physiological mechanisms of autonomic nervous systems. Long-term HRV can represent if a person has Dysautonomia or not as well as the health condition of the heart functions.

The autonomic nervous system is a part of the peripheral nervous system, and controls the functions of organs. The autonomic nervous system is divided into two types: the sympathetic nervous system and the parasympathetic nervous system. The sympathetic nervous system is dominant when pressure exists, and prepares the body while facing pressure and consumes energy. On the contrary, the parasympathetic nervous system is dominant while resting and convalescing, and accelerates and regulates processes such as digestion and growth. In order to let the body rest and convalesce, its activities can preserve energy.

Currently, the most effective method for evaluating the activities of the autonomic nervous system is through the analysis of HRV. The characteristics of HRV vary according to the interaction and regulation of the sympathetic and parasympathetic nervous systems. Thereby, medically, HRV is used to study the regulation of autonomic nervous systems. That is to say, HRV can be used to judge if a person's autonomic nerves are disordered. In addition, HRV can also represent the health of the heart: low HRV means high risk in heart disease. Accordingly, the HRV characteristics can be used to judge or treat multiple diseases, such as arrhythmia, diabetes, and melancholia.

The arrhythmia means abnormal heartbeats, including variations in heartbeat intervals and too fast or too slow heart rates. In addition to arrhythmia due to heart diseases, changes in breathing cause arrhythmia as well. For example, when a person inhales deeply, his heart rate will increase; when he exhales, his heart rate will decrease. These are normal physiological phenomena. Besides, when one exercises, his heart rate increases; when he rests or sleeps, the heart rate decreases. Furthermore, heart rate and its rhythm also change owing to excitement of autonomic nervous system, stimulation by coffee or tea, fever, nervousness, pressure, pain, anoxia, medicine.

When arrhythmia occurs, the symptoms can be none or slight such as feeling acceleration of heartbeats or irregular heartbeats. The symptoms can also be as severe as shock, faint, or even sudden death. Many sudden death patients exhibit no symptoms. Sudden death can even happen to young people. It is regarded in the medical field that in addition to analysis of past cases, sudden lowering of HRV can be used as a predictive indication of diseases. Especially, for busy people, by monitoring of long-term HRV, if the HRV is too low or is lowering gradually, they should take rest immediately for reducing the possibility of sudden death.

HRV can be an indication of the treatment effect for diabetes. In the early phase of diabetes, though the blood sugar is maintained in the normal range, the HRV is lowering gradually. In the middle and last phases of diabetes, the patients can possibly have diabetic neuropathy at the same time. Then the sympathetic and parasympathetic fine fibers start necrotizing. The patients will exhibit dysautonomia symptoms of vertigo (low blood pressure), palpitations, night sweat, and diarrhea. By long-term HRV measurement, it is found that the HRV deviates from original baseline. The treatment effects can be evaluated by the measurement as well.

Moreover, HRV can be used to judge morbidity of melancholia. Melancholia is a medical disease, not just depression only. Tens of millions of people suffer from this disease. Females have twice the possibility of having melancholia than males. Patients of heart disease, paralysis, cancer, and diabetes have higher probability o having melancholia. The HRV of these usually patients exhibits active and low values. According to scientific literature, many prescription drugs of western medicine can improve symptoms of melancholia. According to estimation, 80% to 90% of melancholia patients can be totally cured by professional pharmaceutical therapy and psychotherapy. If long-term HRV is used to trace curative effect, melancholia can be fully healed.

To acquire HRV information, it is not necessary to analyze the details of an electrocardiogram. If the period of heartbeats is given, HRV information can be deduced accordingly. It takes a period of time, around 10 minutes, to measure HRV. It is not possible to know the result in a short time. First, the heartbeat period is given by the electrocardiographic signals. After re-sampling, perform fast Fourier transform to the sampled data for giving the power spectrum of heart rate variability. According to the power spectrum of heart rate variability, the high-frequency (0.15-0.4 Hz) power and low-frequency (0.04-0.15 Hz) power are given. The variation in high- and low-frequency power can be used as the indication of activity of autonomic nerves.

However, in the long-term measurement, if the person under test concentrates in the measurement itself, he might feel nervous or impatient, and thus natural physiological information cannot be given. It is easier to observe problems in heart by long-term measurement. If the measurement is performed during a short term, considering the nervousness or impatience of the person under test, some diseases, such as occasional arrhythmia, cannot be observed.

Accordingly, the present invention provides a measurement apparatus for heart rate variability, which can make the person under test less nervous or less impatient while measuring. Thereby, the real heart rate variability of the person under test can be measured and giving natural heart rate and heart rate variability but not heart rate variability under nervous conditions.

### SUMMARY

An objective of the present invention is to provide a measurement apparatus for heart rate variability, which uses a measurement module for photoplethysmographic (PPG) signal and a measurement module for electrocardiographic (ECG) signal to measure various physiological signals of a human body simultaneously, and thus improving convenience of measuring physiological signals. In addition, the measurement module for PPG signal is set in an earpiece. When the measurement module for PPG signal and the measurement module for ECG signal measure the physiological signals of a person under test, the earpiece can play sound signals for detracting the person under test from the measurement module for PPG signal and the measurement module for ECG signal, and thus eliminating nervousness and impatience of the person under test. Thereby, the real heart rate variability of the person under test can be measured.

The measurement apparatus for heart rat variability according to the present invention comprises an earpiece, a measurement module for PPG signal, a processing module for PPG signal, a measurement module for ECG signal, a processing module for ECG signal, and a control and processing unit. The earpiece is placed into an ear of a person under test. The measurement module for PPG signal is set on one side of the earpiece, and measures the physiological status of the person under test via the ear for producing a first physiological signal. The processing module for PPG signal transmits a sound signal to the earpiece, and receives the first physiological signal for producing a first measured signal according to the first physiological signal. The measurement module for ECG signal measures the physiological status of the person under test and produces a second physiological signal. The processing module for ECG signal receives the second physiological signal and produces a second measured signal according to the second physiological signal. The control and processing unit transmits a sound signal, and receives the first measured signal and the second measured signal for producing a corresponding first waveform diagram and a corresponding second waveform diagram, respectively. Thereby, the earpiece plays the sound signal for detracting the person under test from the measurement module for PPG signal or the measurement module for ECG signal, and thus eliminating nervousness and impatience of the person under test. Accordingly, the real heart rate variability of the person under test can be measured.

### BRIEF DESCRIPTION OF THE DRAWINGS

FIG. 1 shows a block diagram according to a preferred embodiment of the present invention;

FIG. 2 shows a structural schematic diagram of an earpiece for measuring heart rate variability according to a preferred embodiment of the present invention;

FIG. 3 shows a schematic diagram of an earpiece set in an ear according to a preferred embodiment of the present invention; and

FIG. 4 shows a schematic diagram of performing photoplethysmography according to a preferred embodiment of the present invention.

### DETAILED DESCRIPTION

In order to make the structure and characteristics as well as the effectiveness of the present invention to be further understood and recognized, the detailed description of the present invention is provided as follows along with preferred embodiments and accompanying figures.

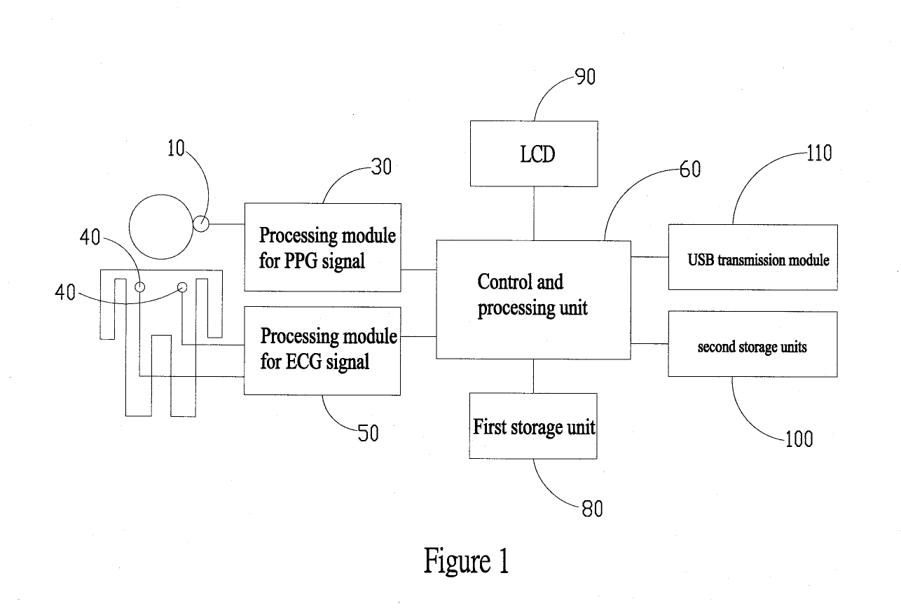

FIG. 1 shows a block diagram according to a preferred embodiment of the present invention. As shown in the figure, the measurement apparatus for heart rat variability according to the present invention comprises an earpiece 10, a measurement module for photoplethysmographic (PPG) signal 20, a processing module for PPG signal 30, a measurement module for electrocardiographic (ECG) signal 40, a processing module for ECG signal 50, and a control and processing unit 60. The earpiece 10 is placed into an ear 70 of a person under test. The measurement module for PPG signal 20 is set on one side of the earpiece 10, and measures the physiological status of the person under test via the ear 70 for producing a first physiological signal. The processing module for PPG signal 30 transmits a sound signal to the earpiece 10, and receives the first physiological signal for producing a first measured signal according to the first physiological signal. The measurement module for ECG signal 40 measures the physiological status of the person under test and produces a second physiological signal. The processing module for ECG signal 50 receives the second physiological signal and produces a second measured signal according to the second physiological signal. The control and processing unit 60 transmits a sound signal, and receives the first measured signal and the second measured signal for producing a corresponding first waveform diagram, similar to the sine wave of heartbeats, and a corresponding second waveform diagram, similar to an electrocardiograph, respectively. Thereby, the earpiece 10 plays the sound signal for detracting the person under test from the measurement module for PPG signal 20 or the measurement module for ECG signal 40, and thus eliminating nervousness and impatience of the person under test. Accordingly, the real heart rate variability of the person under test can be measured.

The present invention further comprises a first storage unit 80, which is coupled to the control and processing unit 60. The control and processing unit 60 stores the first and the second measured signals to the first storage unit 80. The first storage unit 80 is a Compact Flash (CF) card. CF cards have the advantages of high storage capacity, small size, high performance, and convenient portability. In addition, they have fast access time and are compatible with multiple computer operating systems. Thereby, CF cards are widely adopted for data recording in data collection and for accessing data between PCs. A CF card includes a controller, a flash memory array, and an access buffer. The embedded intelligent controller greatly simplifies the design of peripheral circuitry. Besides, it also complies with the interface regulations required by PCMCIA (Personal Computer Memory Card International Association) and ATA (Advanced Technology Attachment). The structure of the buffer in a CF card enables the controller therein to access flash memories while communicating with external equipments. This feature increases reliability of data access in a CF card as well as increasing data transmission rate. CF cards support multiple interface modes, including the Memory Mapped mode and the I/O Card mode of PCMCIA, and the True IDE mode of ATA. When powering on, if the OE pin is low, the CF card enters True IDE mode. Then the OE pin is called ATA SEL. On the contrary, if the OE pin is high when powering on, the CF card enters the PCMCIA mode, namely, the Memory Mapped mode or the I/O Card mode. The corresponding modes can be entered by modifying the configuration register.

In addition, the present invention further comprises a liquid-crystal display (LCD) 90, which is coupled to the control and processing unit 60. The control and processing unit 60 transmits the first waveform diagram and the second waveform diagram to the LCD 90 for displaying. The LCD 90 according to the present invention adopts a thin-film transistor liquid-crystal display (TFT-LCD). The TFT-LCD panel can be regarded as a layer of liquid crystal sandwiched between two glass substrates. The top glass substrate is bonded with a color filter, while the bottom glass substrate has transistors thereon. When current passes through the transistors and produces changes in electric field, the liquid-crystal molecules rotates and thereby changes polarity of light. Then the polarizer is used for determining brightness if a pixel. In addition, because bonding between the top glass substrate and the color filter, each pixel has three colors including red, blue, and green, respectively. These pixels emitting red, blue, and green lights form the image of the panel.

The present invention further comprises one or more second storage units 100, which stores multimedia data such as MP3 data. The control and processing unit 60 reads the multimedia data, converts it, and transmits voice signals. Moreover, the present invention further comprises a USB transmission module 110. The control and processing unit 60 transmits the first and second measured signals to the USB transmission module 110 for sending to a computer. The given timing and pulse data and the analyzed heart rate variability data can be displayed by using Borland C++ Builder for editing user interface windows. Besides, the USB transmission module 110 can transmits data between the computer and the first storage unit 80 as well.

The control and processing unit 60 is mainly used for MP3 encoding/decoding and compression/decompression of other audio formats (such as WMA) for digital media players. After the music is played, the audio data stored in the second storage unit 100 will be played for every 130 ms. In addition, while processing the first and second measured signal, the music will not be interrupted.

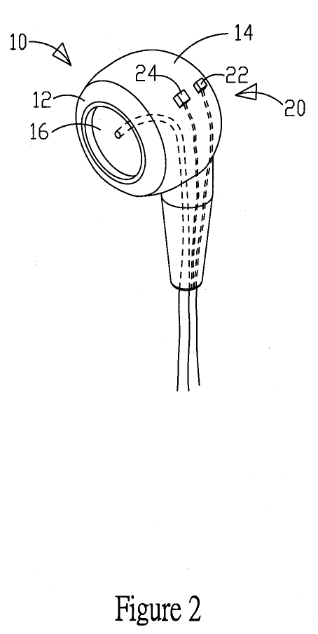

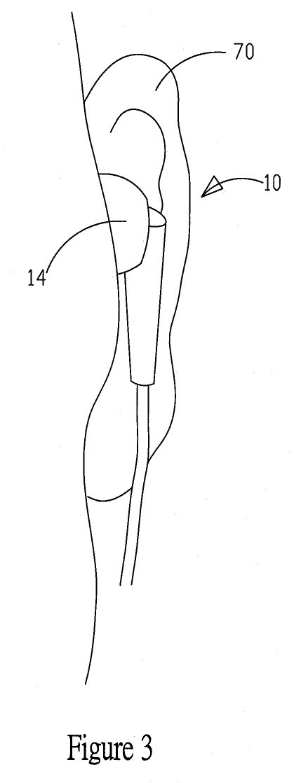

FIG. 2 shows a structural schematic diagram of an earpiece for measuring heart rate variability according to a preferred embodiment of the present invention. In addition, FIG. 3 shows a schematic diagram of an earpiece set in an ear according to a preferred embodiment of the present invention. As shown in the figures, the measurement module for PPG signal 20 according to the present invention includes a light source 22 and a photodetector 24. The light source 22 is set on one side of the earpiece 10. The light source 22 illuminates the skin of the ear 70 and produces reflection light. The photodetector 24 is set on the earpiece 10 and is located on the same side of the light source 22. The photodetector 24 receives the reflection light, produces the first physiological signal according to the reflection light, and transmits the first physiological signal to the processing module for PPG signal 30.

The earpiece 10 according to the present invention includes an embedded part 12 and a holding part 14. The embedded part 12 is placed into the ear 70. The earpiece 10 has a speaker 16, which is set in the embedded part 12. The holding part 14 is set on one side of the embedded part 12. The light source 22 and the photodetector 24 are set in the holding part 14. When the earpiece 10 plays music for the person under test, the light source 22 and the photodetector 24 in the earpiece 10 are used for measuring the HRV of the person under test. Thereby, attention of the person under test can be detracted from the HRV measurement apparatus, and thus eliminating nervousness and impatience of the person under test. Hence, the real heart rate variability of the person under test can be measured.

The present invention adopts photoplethysmography (PPG) to extract the first physiological signal. According to the method, a light source 22 with a red LED is needed and a photodetector 24 of light-receiving transistor is used as the probe of PPG The light source 22 includes red light, and can be a red LED with wavelength 640 nm. The photodetector 24 includes a light-receiving transistor. Because the volume of the light-receiving transistor is relatively small, the photodetector 24 and the light source 22 are set in the earpiece 10. It is uneasy for he person under test to aware the location of the probe of PPG Thereby, when the person under test is testing and listening to the music, his nervousness can be eliminated. Accordingly, the log-term HRV data of the person under test can be measured with better measurement accuracy.

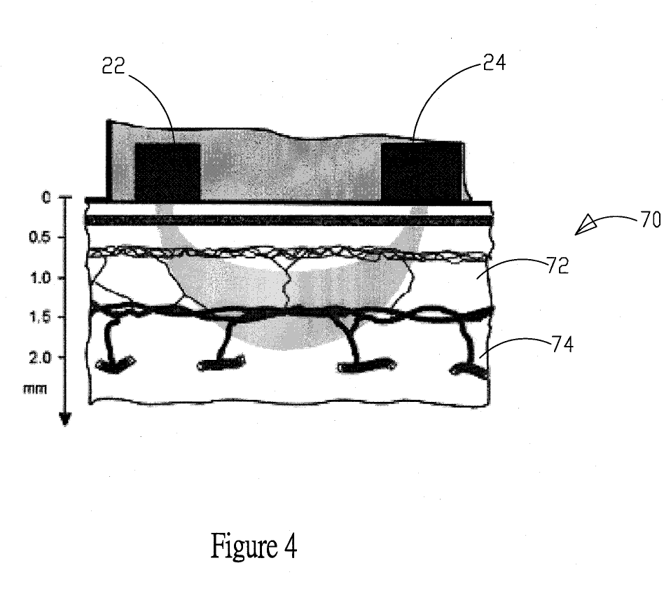

FIG. 4 shows a schematic diagram of performing photoplethysmography (PPG) according to a preferred embodiment of the present invention. As shown in the figure, PPG measures light characteristics by emitting near infrared light source 22 into a selected skin area. When light propagates in the tissue of an organism, it will be absorbed by various absorbing materials such as skin, bones, blood in the artery and the vein. Besides, the artery vessels contain more blood in the systolic period than in the diastolic period. The radii of artery vessels increase as the blood pressure increases, which increase happens only to arteries and arterioles but not to veins. During the systolic period, the absorbability of light increases due an increase of light absorbing materials (such as hemoglobin) as well as an increase of distance traveled by light in the arteries. For the overall absorbability, it acts like an alternating current (AC) component. The AC component helps to identify invariant quantity in the vein blood and in the artery blood, and to identify the difference between light absorbability without pulse component (DC component) such as skin and light absorbability with a pulse component (AC component). The AC component will not exceed 1%-2% of the DC component. Thereby, PPG refers to receiving waveforms of light signals changing according to time and organism variations. According to the present invention, the light source 22 is emitted to the epidermis 72 of the ear 70. The light passing through the epidermis 72 will then be reflected by the derma 74.

To sum up, the present invention relates to a measurement apparatus for heart rate variability, which comprises an earpiece, a measurement module for photoplethysmographic (PPG) signal measuring an ear and producing a first physiological signal of a person under test, a measurement module for electrocardiographic (ECG) signal measuring a second physiological signal of the person under test, and a control and processing unit transmitting a sound signal to the earpiece. By playing the sound signal from the earpiece, the attention of the person under test can be detracted from the measurement module for PPG signal or the measurement module for ECG signal. Thereby, nervousness and impatience of the person under test can be eliminated, and hence the real heart rate variability of the person under test can be measured.

Accordingly, the present invention conforms to the legal requirements owing to its novelty, non-obviousness, and utility. However, the foregoing description is only a preferred embodiment of the present invention, not used to limit the scope and range of the present invention. Those equivalent changes or modifications made according to the shape, structure, feature, or spirit described in the claims of the present invention are included in the appended claims of the present invention.

1. A measurement apparatus for heart rat variability, comprising:
an earpiece, placed into an ear of a person under test;
a measurement module for photoplethysmographic (PPG) signal, set on one side of the earpiece, measuring the physiological status of the person under test via the ear, and producing a first physiological signal;
a processing module for PPG signal, transmitting a sound signal to the earpiece, receiving the first physiological signal, and producing a first measured signal according to the first physiological signal;
a measurement module for electrocardiographic (ECG) signal, measuring the physiological status of the person under test, and producing a second physiological signal;
a processing module for ECG signal, receiving the second physiological signal, and producing a second measured signal according to the second physiological signal; and
a control and processing unit, transmitting a sound signal, receiving the first measured signal and the second measured signal, and producing a corresponding first waveform diagram and a corresponding second waveform diagram, respectively.

2. The measurement apparatus for heart rate variability of claim 1, wherein the measurement module for PPG signal comprises:
a light source, set on one side of the earpiece, illuminating the skin of the ear, and producing reflection light; and
a photodetector, set on the earpiece and on the same side of the light source, receiving the reflection light, producing the physiological signal according to the reflection light, and transmitting the first physiological signal to the processing module for PPG signal.

3. The measurement apparatus for heart rate variability of claim 2, wherein the light source includes red light.

4. The measurement apparatus for heart rate variability of claim 3, wherein the wavelength of the red light is 640 nm.

5. The measurement apparatus for heart rate variability of claim 2, wherein the light source is a red-light LED.

6. The measurement apparatus for heart rate variability of claim 2, wherein the photodetector includes a light-receiving transistor.

7. The measurement apparatus for heart rate variability of claim 2, wherein the light source passes through the epidermis of the ear to the derma, and the derma reflects the light and produces the reflection light.

8. The measurement apparatus for heart rate variability of claim 2, wherein the earpiece has a speaker.

9. The measurement apparatus for heart rate variability of claim 8, wherein the earpiece comprises:
an embedded part, placed into the ear, and holding the speaker; and
a holding part, set on one side of the embedded part, and holding the light source and the photodetector.

10. The measurement apparatus for heart rate variability of claim 1, and further comprising a first storage unit, coupled to the control and processing unit, and storing the first measured signal and the second measured signal.

11. The measurement apparatus for heart rate variability of claim 10, wherein the first storage unit includes a Compact Flash (CF) card.

12. The measurement apparatus for heart rate variability of claim 1, and further comprising one or more second storage units, storing multimedia data, and the control and processing unit reading and converting the multimedia data and playing the sound signal.

13. The measurement apparatus for heart rate variability of claim 1, and further comprising a liquid crystal display (LCD), coupled to the control and processing unit, and the control and processing unit transmitting the first waveform diagram and the second waveform diagram to the LCD for displaying.

14. The measurement apparatus for heart rate variability of claim 13, wherein the LCD is a thin-film transistor liquid-crystal display (TFT-LCD).

15. The measurement apparatus for heart rate variability of claim 1, and further comprising a USB transmission module, the control and processing unit transmitting the first measured signal and the second measured signal to the USB transmission module for further transmitting to a computer.
